(19) United States
(12) Patent Application Publication
(10) Pub. No.: US 2004/0009899 A1
(43) Pub. Date: Jan. 15, 2004

(54) Treating dominant disorders

(76) Inventors: Cynthia T. McMurray, Oronoco, MN (US); Elliott Richelson, Ponte Vedra Beach, FL (US); Beth M. McMahon, Alameda, CA (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/195,722

(22) Filed: Jul. 15, 2002

(51) Int. Cl. A61K 48/00; C07H 21/02

(52) U.S. Cl. 514/8; 514/44; 536/23.1

(57) ABSTRACT

This invention provides methods and materials for reducing the level of an RNA or polypeptide expressed by a mutant allele of a gene that causes a dominant disorder in a mammal. The methods include administering a PNA oligomer to a mammal that is heterozygous for such a mutant allele. By using these methods, the level of an RNA or polypeptide encoded by the mutant allele is reduced to a greater extent than the level of an RNA or polypeptide encoded by the non-mutant allele.

### BACKGROUND

1. Technical Field

This invention relates to treatment of autosomal dominant disorders, and particularly relates to treatment of such disorders with polyamide nucleic acid oligomers.

2. Background Information

Polyamide nucleic acid (PNA; also known as peptide nucleic acid) oligomers are modified oligonucleotides in which the phosphodiester backbone of the oligonucleotide is replaced with a neutral polyamide backbone consisting of N-(2-aminoethyl)glycine units linked through amide bonds (FIG. 1). See, e.g., Nielsen et al. (1991) Science 254:1497-1500, and Nielsen et al. (1994) Bioconjugate Chem. 5:3-7.

PNA oligomers bind to complementary DNA or RNA by standard Watson-Crick base pairing rules (Wittung et al. (1994) Nature 368:561-563). PNA oligomers can bind both DNA and RNA to form PNA/DNA and PNA/RNA duplexes. The resulting PNA/DNA or PNA/RNA duplexes have higher melting temperatures and thus are more stable than corresponding DNA/DNA or DNA/RNA duplexes (Egholm et al. (1993) Nature 365:566-568; and Møllegaard et al. (1994) Proc. Natl. Acad. Sci. USA 91:3892-3895). This high degree of thermal stability may be attributed to the lack of charge repulsion due to the neutral backbone in a PNA oligomer. The neutral backbone also results in a PNA/DNA (RNA) duplex with a melting temperature that is practically independent of salt concentration. PNA/DNA duplex interactions therefore offer an advantage over DNA/DNA duplex interactions, which are highly dependent on ionic strength.

In addition to creating high affinity heteroduplexes with DNA and RNA, PNA oligomers also can bind to DNA with increased specificity. When a PNA/DNA duplex mismatch is melted, there is an 8° C. to 20° C. decrease in melting temperature as compared to a corresponding DNA/DNA duplex. This magnitude of a drop in melting temperature is not observed when the corresponding DNA/DNA duplex contains a mismatch.

In addition to binding to DNA and RNA with greater affinity and specificity than DNA oligonucleotides, PNA oligomers have non-natural (polyamide) backbones that are not recognized by either nucleases or proteases (Demidov et al. (1994) Biochem. Pharmacol. 48:1310-1313). PNA oligomers therefore are more resistant than standard oligonucleotides and oligopeptides to enzymatic degradation.

### SUMMARY

The invention provides methods and materials for reducing the level of RNA or polypeptide expressed from mutant alleles of genes that cause dominant disorders. The methods involve administering a PNA oligomer to a mammal that is heterozygous for such a mutant allele. By using these methods, the level of RNA or polypeptide encoded by the mutant allele is reduced to a greater extent than the level of RNA or polypeptide encoded by the non-mutant allele.

The PNA oligomers used in the methods provided herein can be designed based on sequence information about the mutant allele, wherein the sequence information is obtained by, for example, a method involving polymerase chain reaction (PCR; e.g., linear amplification sequencing of a PCR-amplified genomic fragment). PNA oligomers can be delivered to a mammal diagnosed with a dominant disorder such as Huntington disease (HD). By reducing the level of RNA or polypeptide expressed from the disease-associated allele, a PNA oligomer can alleviate the symptoms and reverse the pathophysiology of the disease. PNA oligomers therefore are useful for treatment of any of a number of autosomal dominant disorders.

The invention is based on the discovery that PNA oligomers can be specifically directed against a mutant allele of a disease gene, without affecting the corresponding non-mutant allele. Specifically, the invention is based on the discovery that a PNA oligomer directed against an HD-associated allele (a mutant human HD allele) can reverse the pathophysiology of the disease. When administered to an animal that is heterozygous for the mutant allele, a specifically directed PNA can reduce the expression of RNA and polypeptide from the mutant allele while having little or no effect on expression from the corresponding non-mutant allele. As a result, a PNA oligomer can promote increased survival, improved motility and motor skills, reduced clasping phenotype, stabilization of body weight, improved grooming, increased brain weight, and reduced incidence of nuclear inclusions. Methods of the invention therefore are useful for treating animals having diseases such as HD, as well as other autosomal dominant disorders. These methods involve administering to an affected mammal one or more PNA oligomers that are specific for a mutant allele of a disease gene.

The invention features a method for reducing the level of an RNA or the level of a polypeptide in a mammal having a mutant allele that causes a dominant disorder. The RNA and the polypeptide can be encoded by the mutant allele, and the mammal can be heterozygous for the mutant allele. The method can involve administering a polyamide nucleic acid oligomer to the mammal under conditions wherein the level of the RNA is reduced to a greater extent than the reduction, if any, in the amount of a second RNA or the level of the polypeptide is reduced to a greater extent than the reduction, if any, in the amount of a second polypeptide. The second RNA and second polypeptide can be encoded by a second allele in the mammal, wherein the second allele corresponds to the mutant allele and does not cause the dominant disorder. The mammal can be a human. The dominant disorder can be an autosomal dominant disorder (e.g., Huntington disease). The RNA can be mRNA. The polyamide nucleic acid oligomer can be administered into the brain of the mammal, or can be administered intraperitoneally to the mammal. The polyamide nucleic acid oligomer can contain a sequence having specificity for a transcription initiation site of the mutant allele, a translation initiation site of the mutant allele, or a region between the transcription initiation site and the translation initiation site of the mutant allele. The polyamide nucleic acid oligomer can have the sequence set forth in SEQ ID NO:3.

In another aspect, the invention features a method for treating a dominant disorder caused by a mutant allele in a mammal. The method can involve obtaining a polyamide nucleic acid oligomer based on sequence information obtained from the mammal, wherein the polyamide nucleic acid oligomer has specificity for the mutant allele. The method also can involve administering the polyamide nucleic acid oligomer to the mammal under conditions wherein expression of a first polypeptide is reduced such that the amount of reduction in expression of the first polypeptide is greater than the amount of reduction, if any, in expression of a second polypeptide. The first polypeptide can be encoded by the mutant allele and the second polypeptide can be encoded by a second allele in the mammal, wherein the second allele corresponds to the mutant allele and does not cause the dominant disorder. The mammal can be a human. The dominant disorder can be an autosomal dominant disorder (e.g., Huntington disease). The sequence information can be obtained by PCR. The polyamide nucleic acid oligomer can be administered into the brain of the mammal, or can be administered intraperitoneally to the mammal. The polyamide nucleic acid oligomer can contain a sequence having specificity for a transcription initiation site of the mutant allele, a translation initiation site of the mutant allele, or a region between the transcription initiation site and the translation initiation site of the mutant allele. The polyamide nucleic acid oligomer can contain the sequence set forth in SEQ ID NO:3.

In another aspect, the invention features a method for treating a dominant disorder caused by a mutant allele in a mammal. The method can include (a) obtaining at least a portion of the sequence of the mutant allele; (b) obtaining a polyamide nucleic acid oligomer based on the sequence, wherein the polyamide nucleic acid oligomer has specificity for the mutant allele; and (c) administering the polyamide nucleic acid oligomer to the mammal. The polyamide nucleic acid oligomer can be administered under conditions wherein expression of a first polypeptide is reduced, such that the amount of reduction in expression of the first polypeptide is greater than the amount of reduction, if any, in expression of a second polypeptide. The first polypeptide can be encoded by the mutant allele and the second polypeptide can be encoded by a second allele in the mammal, wherein the second allele corresponds to the mutant allele and does not cause the dominant disorder. The mammal can be a human. The dominant disorder can be an autosomal dominant disorder (e.g., Huntington disease). The sequence can be obtained by PCR. The polyamide nucleic acid oligomer can be administered into the brain of the mammal, or can be administered intraperitoneally to the mammal. The polyamide nucleic acid oligomer can contain a sequence having specificity for a transcription initiation site of the mutant allele, a translation initiation site of the mutant allele, or a region between the transcription initiation site and the translation initiation site of the mutant allele. The polyamide nucleic acid oligomer can contain the sequence set forth in SEQ ID NO:3.

In yet another aspect, the invention features a method of assisting a medical professional in treating a dominant disorder caused by a mutant allele in a mammal. The method can include providing a polyamide nucleic acid oligomer based on sequence information obtained from the mammal, wherein the polyamide nucleic acid oligomer has specificity for the mutant allele. Administration of the polyamide nucleic acid oligomer can reduce expression of a first polypeptide in the mammal such that the amount of reduction in expression of the first polypeptide is greater than the amount of reduction, if any, in expression of a second polypeptide. The first polypeptide can be encoded by the mutant allele and the second polypeptide can be encoded by a second allele in the mammal, wherein the second allele corresponds to the mutant allele and does not cause the dominant disorder. The mammal can be a human. The dominant disorder can be an autosomal dominant disorder (e.g., Huntington disease). The sequence information can be obtained by PCR. The polyamide nucleic acid oligomer can contain a sequence having specificity for a transcription initiation site of the mutant allele, a translation initiation site of the mutant allele, or a region between the transcription initiation site and the translation initiation site of the mutant allele. The polyamide nucleic acid oligomer can contain the sequence set forth in SEQ ID NO:3.

The invention also features a method of assisting a medical professional in treating multiple different mammals, wherein each of the multiple different mammals has a dominant disorder caused by a mutant allele. The method can include providing a plurality of different polyamide nucleic acid oligomers based on sequence information obtained from each of the multiple different mammals, wherein at least one of the plurality of different polyamide nucleic acid oligomers has specificity for the mutant allele from each of the multiple different mammals. Administration of the at least one of the plurality of different polyamide nucleic acid oligomers to each of the multiple different mammals can reduce expression of a first polypeptide in each of the multiple different mammals such that the amount of reduction in expression of the first polypeptide is greater than the amount of reduction, if any, in expression of a second polypeptide. The first polypeptide can be encoded by the mutant allele and the second polypeptide can be encoded by a second allele in each of the multiple different mammals, wherein the second allele corresponds to the mutant allele and does not cause the dominant disorder. Each of the multiple different mammals can be a human. The dominant disorder can be an autosomal dominant disorder (e.g., Huntington disease). The sequence information can be obtained by PCR. Each of the plurality of polyamide nucleic acid oligomers can contain a sequence having specificity for a transcription initiation site of the mutant allele, a translation initiation site of the mutant allele, or a region between the transcription initiation site and the translation initiation site of the mutant allele.

In another aspect, the invention features a method for reducing the level of an RNA or the level of a polypeptide in a mammal having a mutant allele that causes a dominant disorder, wherein the RNA and the polypeptide are encoded by the mutant allele, and wherein the mammal is heterozygous for the mutant allele. The method can involve administering at least two polyamide nucleic acid oligomers to the mammal under conditions wherein the level of the RNA is reduced to a greater extent than the reduction, if any, in the amount of a second RNA, or the level of the polypeptide is reduced to a greater extent than the reduction, if any, in the amount of a second polypeptide. Each of the at least two polyamide nucleic acid oligomers can have a different sequence. The second RNA and the second polypeptide can be encoded by a second allele in the mammal, wherein the second allele corresponds to the mutant allele and does not cause the dominant disorder. The mammal can be a human. The dominant disorder can be an autosomal dominant disorder (e.g., Huntington disease). The RNA can be mRNA. Each of the at least two polyamide nucleic acid oligomers can be administered into the brain of the mammal, or can be administered intraperitoneally to the mammal. Each of the at least two polyamide nucleic acid oligomers can contain a sequence having specificity for a transcription initiation site of the mutant allele, a translation initiation site of the mutant allele, or a region between the transcription initiation site and the translation initiation site of the mutant allele.

In yet another aspect, the invention features a method for reducing the level of an RNA or the level of a polypeptide in a mammal having a mutant allele that causes a dominant disorder, wherein the RNA and the polypeptide are encoded by the mutant allele, and wherein the mammal is heterozygous for the mutant allele. The method can include administering to the mammal between 0.05 mg and 0.5 mg of a polyamide nucleic acid oligomer per kg of body weight of the mammal. The administration can be under conditions wherein the level of the RNA is reduced to a greater extent than the reduction, if any, in the amount of a second RNA, or the level of the polypeptide is reduced to a greater extent than the reduction, if any, in the amount of a second polypeptide. The second RNA and the second polypeptide can be encoded by a second allele in the mammal, wherein the second allele corresponds to the mutant allele and does not cause the dominant disorder. The mammal can be a human. The dominant disorder can be an autosomal dominant disorder (e.g., Huntington disease). The RNA can be mRNA. The polyamide nucleic acid oligomer can be administered into the brain of the mammal, or can be administered intraperitoneally to the mammal. The polyamide nucleic acid oligomer can contain a sequence having specificity for a transcription initiation site of the mutant allele, a translation initiation site of the mutant allele, or a region between the transcription initiation site and the translation initiation site of the mutant allele. The polyamide nucleic acid oligomer can contain the sequence set forth in SEQ ID NO:3.

In another aspect, the invention features a kit for assisting a medical professional in treating multiple different mammals, wherein each of the multiple different mammals has a dominant disorder caused by a mutant allele. The kit can include a plurality of polyamide nucleic acid oligomers, wherein the sequence of each of the plurality of polyamide nucleic acid oligomers is different and based on sequence information obtained from each of the multiple different mammals. At least one of the plurality of polyamide nucleic acid oligomers can have specificity for the mutant allele from each of the multiple different mammals, such that administration of at least one of the plurality of polyamide nucleic acid oligomers to each of the multiple different mammals can reduce expression of a first polypeptide in each of the multiple different mammals. The amount of reduction in expression of the first polypeptide can be greater than the amount of reduction, if any, in expression of a second polypeptide. The first polypeptide can be encoded by the mutant allele and the second polypeptide can be encoded by a second allele in each of the multiple different mammals, wherein the second allele corresponds to the mutant allele and does not cause the dominant disorder. Each of the multiple different mammals can be a human. The dominant disorder can be an autosomal dominant disorder (e.g., Huntington disease). The sequence information can be obtained by PCR. Each of the plurality of polyamide nucleic acid oligomers can contain a sequence having specificity for a transcription initiation site of the mutant allele, a translation initiation site of the mutant allele, or a region between the transcription initiation site and the translation initiation site of the mutant allele.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a diagram of an interaction between a PNA oligomer and a DNA oligomer.

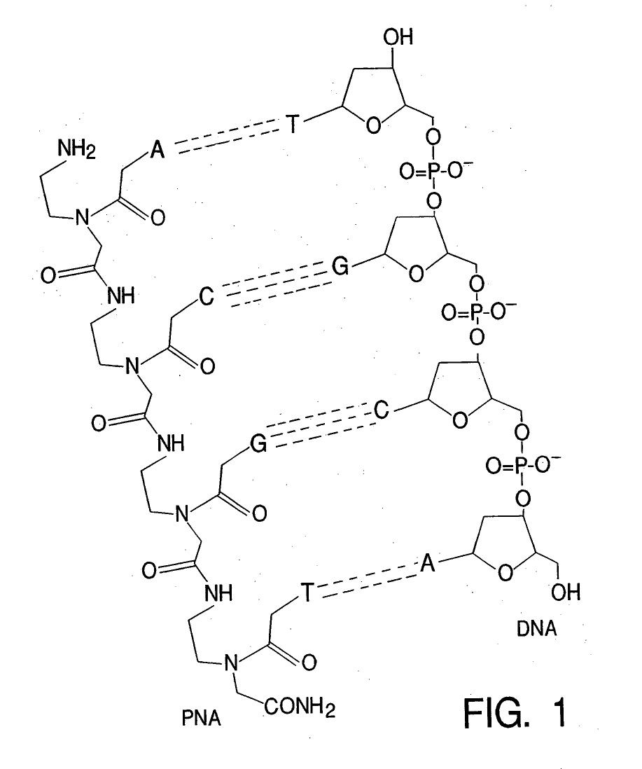

FIG. 2 is a diagram of an alignment of human and mouse HD gene sequences from the R6/1 transgenic mouse (SEQ ID NO:1 and SEQ ID NO:2, respectively). This alignment was used to design the PNA oligomers that target a mutant HD allele and not a non-mutant HD allele. The arrow indicates the translational start codon. Asterisks indicate nucleotide residues that are conserved between the homologues. The “HD sense” (HDs) PNA sequence is underlined (5′-GGACTGCCGTGCCG-3′; SEQ ID NO:3).

FIG. 3 is a photograph of a western immunoblot. Striatal tissue (left panels) and liver tissue (right panels) from R6/1 transgenic mice and control mice were evaluated for expression of the htt polypeptide from the human HD transgene and from the endogenous mouse Hdh gene. Mice were treated for 4 or 6 days with either a random PNA control (“Ran,” denoted herein as “HDscr”) or the HDs PNA oligomer targeted to the human HD transgene. The 1C2 antibody (top panels) was used to detect mutant htt polypeptides with expanded glutamine repeats, while the 2166 antibody (middle panels) was used to detect the full-length mouse htt polypeptide. GAPDH levels were detected as a control.

FIG. 4 is a graph plotting the average body weights of treated (PNA) and untreated (ACSF) transgenic (R6/2) and non-transgenic (NTG) mice at various timepoints.

FIG. 5 is a graph plotting the motility of treated (PNA) and untreated (ACSF) transgenic (R6/2) and non-transgenic (NTG) mice at various times.

FIG. 6 is a column graph plotting the average weights of brain hemispheres from R6/2 mice treated with various amounts of the HDs PNA oligomer, untreated R6/2 mice, or nontransgenic B6CBA control mice.

### DETAILED DESCRIPTION

This invention provides methods and materials for reducing the level of RNA or polypeptide expressed from a mutant allele that causes a dominant disorder in a mammal. The methods involve administering one or more PNA oligomers to a mammal that is heterozygous for such a mutant allele. By using these methods, the level of RNA or polypeptide encoded by the mutant allele is reduced to a greater extent than the level of RNA or polypeptide encoded by the non-mutant allele. As used herein, the term “mutant allele” refers to an allele that is associated with a disease, while “non-mutant allele” refers to an allele that is not associated with the disease. An allele can be identified as a mutant allele, for example, if its nucleotide sequence can be linked to a certain disease. For example, HD alleles that have expanded CAG repeat tracts are known to be associated with HD, and thus can be termed “mutant alleles”. A non-mutant allele can have the nucleotide sequence of the wild type allele or can have a nucleotide sequence that differs from that of the wild type allele but that is not associated with the disease. An HD allele without an expanded CAG repeat tract, for example, is a non-mutant allele.

1. PNA Oligomers and Compositions Containing PNA Oligomers

A PNA oligomer is a modified oligonucleotide in which the phosphodiester backbone of the oligonucleotide is replaced with a polyamide backbone consisting of N-(2-aminoethyl)glycine units linked through amide bonds. Any method can be used to make a PNA oligomer. Typically, PNA oligomers are made as described elsewhere (see, e.g., U.S. Pat. No. 5,539,082). A PNA oligomer can be an antisense or a sense PNA oligomer. The term “antisense PNA oligomer” refers to any PNA oligomer having sequence specificity for an RNA molecule found within a cell. The term “sense PNA oligomer” refers to any PNA oligomer having sequence specificity for a region of nucleic acid from a strand that can be used as the template strand during transcription, including reverse transcription. Sense PNA oligomers also are referred to as “anti-gene” PNA oligomers. It is noted that sequence specificity is based on complementarity with respect to an anti-parallel orientation.

The PNA oligomers provided herein have specificity for target sequences within particular nucleic acid molecules. As used herein, the “specificity” of a PNA oligomer for a particular target sequence means that the PNA oligomer binds to the target sequence in a manner that is dependent on the sequence of the target nucleic acid. A PNA oligomer can be completely complementary across its length to a target sequence. Alternatively, a PNA oligomer can contain mismatches, deletions, or extra PNA monomers, provided that the PNA oligomer can bind to its target sequence.

The process of “targeting” a PNA oligomer to a particular nucleic acid sequence usually begins with the identification of a nucleic acid whose function is to be modulated. This nucleic acid sequence can be, for example, a cellular gene (or mRNA transcribed from a gene) whose expression is associated with a particular disorder or disease state. For example, a PNA oligomer can be targeted specifically to the mutant allele of a gene (e.g., a mutant HD allele) in a heterozygous individual, such that the PNA oligomer will not affect the corresponding non-mutant allele of the gene.

The targeting process also includes the identification of a site or sites within the target nucleic acid molecule where an interaction can occur such that the desired effect, e.g., modulation of gene expression, will result. Target sites for PNA oligomers can include regions at or near the transcription initiation site, or at or near the translation initiation site or translation stop site of the open reading frame (ORF) of a gene. In addition, the ORF can be targeted by a PNA oligomer, as can the 5′ or 3′ untranslated regions. Furthermore, PNA oligomers can be directed at intron regions or intron-exon junction regions. PNA oligomers directed to transcription initiation sites, translation initiation sites, or regions between transcription and translation initiation sites are particularly useful.

PNA oligomers can be designed based on sequence information obtained from the nucleic acid to be targeted. Typically, the sequence of a targeted nucleic acid (e.g., the mutant allele of a disease gene) is compared with the sequence of a non-targeted nucleic acid that corresponds to the non-mutant allele of the same gene. Such comparison can reveal nucleotide sequence variations between the two alleles. The sequence information can be obtained using any of a number of methods, including those known in the art. Suitable methods for obtaining sequence information include, for example, standard nucleic acid sequence techniques as well as PCR techniques (e.g., linear amplification sequencing of PCR-amplified genomic fragments). When targeting an mRNA sequence, PNA oligomers can be directed to regions that are most accessible, for example, regions predicted to be at or near the surface of the mRNA molecule.

PNA oligomers can be obtained commercially from, for example, PerSeptive Biosystems (Framingham, Mass., USA). Alternatively, PNA oligomers can be synthesized manually from PNA monomers (see, e.g., Norton J. C. (1995) Bioorg. Med. Chem. 3:437-445; and Cory D. R. (1997) Trends in Biotech. 15:224-229). PNA oligomers can have any nucleobase sequence determined to be useful for reducing expression from a mutant allele. Furthermore, PNA oligomers can be any length provided that they contain at least two PNA monomers. PNA oligomers that are useful in methods of the invention typically contain between 10 and 50 nucleobase residues (e.g., 10, 11, 12, 13, 14, 15, 20, 25, 30, 40, or 50 nucleobase residues). A PNA oligomer that is 14 residues in length and has the sequence 5′-GGACTGCCGTGCCG-3′ (SEQ ID NO:3), for example, is particularly useful for reducing expression from an expanded (i.e., mutant) human HD allele (see Examples 3 and 4).

PNA oligomers can be formulated for administration to a mammal (e.g., a mouse, a dog, a cat, a horse, a cow, or a human). Methods for formulating and subsequently administering therapeutic compositions are well known to those skilled in the art. Dosages typically are dependent on the severity and responsiveness of the disease state to be treated, with the course of treatment lasting from several days to several months, or until a cure is effected or a diminution of the disease state is achieved. Standard pharmacological studies can be used to determine optimum dosages, dosing methodologies, and repetition rates. Optimum dosages can vary depending on the relative potency of individual PNA oligomers, and generally can be estimated based on the EC₅₀ found to be effective using in vitro and/or in vivo animal models. Typically, dosage is from 0.01 μg to 100 g per kg of body weight (e.g., from 1 μg to 100 mg, from 10 μg to 10 mg, or from 50 μg to 500 μg per kg of body weight). PNA oligomers may be given once or more daily, weekly, or even less often. An individual may require maintenance therapy to prevent recurrence of the disease state.

PNA oligomers can be admixed, encapsulated, conjugated or otherwise associated with other molecules, molecular structures, or mixtures of compounds such as, for example, liposomes, receptor targeted molecules, or oral, rectal, topical or other formulations for assisting in uptake, distribution and/or absorption.

PNA oligomers also can be combined with pharmaceutically acceptable carriers. Pharmaceutically acceptable carriers are pharmaceutically acceptable solvents, suspending agents, or any other pharmacologically inert vehicles for delivering one or more PNA oligomers to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation, water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

Pharmaceutical compositions containing PNA oligomers can be administered by a number of methods, depending upon whether local or systemic treatment is desired and upon the area to be treated. Administration can be, for example, parenteral (e.g., by subcutaneous, intrathecal, intraventricular, intramuscular, or intraperitoneal injection, or by intravenous drip); oral; topical (e.g., transdermal, sublingual, ophthalmic, or intranasal); or pulmonary (e.g., by inhalation or insufflation of powders or aerosols). Administration can be rapid (e.g., by injection) or can occur over a period of time (e.g., by slow infusion or administration of slow release formulations). As described herein, PNA oligomers can be administered systemically (e.g., intravenously, intraperitoneally, or subcutaneously) to reduce the levels of mRNA and/or polypeptide expressed from a mutant allele in the brain. Such PNA oligomers can be administered alone (i.e., without any carriers or other additives), or PNA oligomers can be administered together with agents capable of enhancing penetration of the blood/brain barrier.

Compositions and formulations for parenteral, intrathecal or intraventricular administration can include sterile aqueous solutions (e.g., sterile physiological saline), which also can contain buffers, diluents and other suitable additives (e.g., penetration enhancers, carrier compounds and other pharmaceutically acceptable carriers). Sterile physiological saline is particularly useful.

Compositions and formulations for oral administration can include, for example, powders or granules, suspensions or solutions in water or non-aqueous media, capsules, sachets, or tablets. Such compositions also can contain thickeners, flavoring agents, diluents, emulsifiers, dispersing aids, or binders.

Formulations for topical administration of PNA oligomers can include, for example, sterile and non-sterile aqueous solutions, non-aqueous solutions in common solvents such as alcohols, or solutions in liquid or solid oil bases. Such solutions also can contain buffers, diluents, or other suitable additives. Pharmaceutical compositions and formulations for topical administration can include transdermal patches, ointments, lotions, creams, gels, drops, suppositories, sprays, liquids, and powders. Conventional pharmaceutical carriers, aqueous, powder or oily bases, thickeners and the like may be useful.

Pharmaceutical compositions containing PNA oligomers include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsions often are biphasic systems comprising two immiscible liquid phases that are intimately mixed and dispersed with each other; in general, emulsions are either of the water-in-oil (w/o) or oil-in-water (o/w) variety. Emulsion formulations are particularly useful for oral delivery of therapeutic compositions due to their ease of formulation and efficacy of solubilization, absorption, and bioavailability. Liposomes are vesicles that have a membrane formed from a lipophilic material and an aqueous interior that can contain the composition to be delivered. Liposomes can be particularly useful due to their specificity and the duration of action they offer from the standpoint of drug delivery.

PNA oligomers can be modified to contain charged moieties such that salt forms can be made. For example, several (e.g., two, three, or four) lysine residues can be added to the amino terminal end of a PNA oligomer to enhance its salt characteristics. Such modified PNA oligomers can be formulated into any pharmaceutically acceptable salts, esters, or salts of such esters, or any other compound which, upon administration to a mammal (e.g., a human), is capable of providing (directly or indirectly) the biologically active metabolite or residue thereof. Accordingly, for example, the invention provides pharmaceutically acceptable salts of PNA oligomers adapted to form salts. The term “pharmaceutically acceptable salts” refers to physiologically and pharmaceutically acceptable salts of the PNA oligomers useful in methods of the invention (i.e., salts that retain the desired biological activity of the parent PNA without imparting undesired toxicological effects). Examples of pharmaceutically acceptable salts include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); salts formed with organic acids (e.g., acetic acid, citric acid, oxalic acid, palmitic acid, or fumaric acid); and salts formed with elemental anions (e.g., bromine, iodine, or chlorine).

2. Using PNA Oligomers to Target Mutant Alleles Associated with Dominant Disorders

Autosomally inherited diseases are inherited through the non-sex chromosomes. Dominant inheritance of a disease occurs when a mutant allele from one parent is capable of causing disease even though the allele from the other parent is non-mutant. Autosomal dominant inheritance therefore is marked by the primary feature that one copy of a mutant allele is sufficient for expression of a trait. If one parent has one non-mutant and one mutant allele for an autosomal dominant disease and the other parent has two non-mutant alleles, all offspring have a 50% chance of inheriting the disease.

HD is an autosomal dominant, inherited disorder that displays a progressive neurodegenerative phenotype (Petersen et al. (1999) Exp. Neurol. 157:1-18; Manfredi and Beal (2000) Brain Pathol. 10:462-472; and Vonsattel and DiFiglia (1998) J. Neuropathol. Exp. Neurol. 57:369-384). The disorder is characterized by motor disturbances such as chorea and dystonia, personality changes, and cognitive decline. Pathophysiology is restricted to the brain, with atrophy occurring foremost in the striatum and to a lesser extent in the cortex. The human HD gene (HD) has been identified but the function of the encoded htt polypeptide is unknown. The mouse HD gene (Hdh) also has been identified. The underlying mutation in HD is a CAG repeat expansion that encodes a polyglutamine tract (McMurray (1999) Proc. Natl. Acad. Sci. USA 96:1823-1825). Most data support a role for polyglutamine-induced aggregation and formation of inclusion bodies as a component of pathogenesis (Alves-Rodriguez et al. (1998) Trends Neurosci. 21:516-520). However, the mechanism by which such polyglutamine-containing polypeptides lead to neural cell death remains unclear.

Methods of the invention are particularly useful for treating individuals who are heterozygous for a gene associated with an autosomal dominant disorder. Examples of dominant disorders that can be treated by methods of the invention include, without limitation, Huntington disease, neurofibromatosis, polycystic kidney disease, and certain hereditary cancers (e.g., some inherited breast, ovarian, and colorectal cancers). Other examples include, without limitation, spinocerebellar ataxia (SCA) type 1, SCA type 2, SCA type 3 (Machado-Joseph disease), autosomal dominant juvenile myoclonic epilepsy, and autosomal dominant spastic paraparesis. A PNA oligomer that is targeted to a mutant allele can be administered to a mammal (e.g., a human) that is heterozygous for the allele. Such treatment can result in reduced expression of the mutant allele, with less of an effect (e.g., little or no effect) on expression of the non-mutant allele.

Recent discoveries have provided for in vivo use of PNA oligomers and circumvented the need to couple PNA oligomers to transporter molecules, permeabilize cells before PNA treatment, or microinject PNA oligomers directly into cells. For example, carrier-free PNA oligomers that are injected directly into rat brains can enter neuronal cells and inhibit protein synthesis from the genes to which they are targeted (Tyler et al. (1998) FEBS Lett. 421:280-284). The effects of these PNA oligomers can be reversible and specific. Unmodified PNA oligomers that are administered to rats by intraperitoneal (i.p.) injection can cross the blood-brain barrier and specifically reduce expression from the targeted neurotensin receptor-1 gene (Tyler et al. (1999) Proc. Natl. Acad. Sci. USA 96:753-7058; see also PCT/US98/21888).

Methods of the invention can involve administering a single PNA to a mammal (e.g., a human) that is heterozygous for a mutant allele that is associated with a dominant disorder. Alternatively, a plurality of PNA oligomers can be administered to a mammal. As used herein, a “plurality” of PNA oligomers refers to at least 2 PNA oligomers (e.g., 2, 3, 4, 5, 6, 7, 8, 9, 10, 15, 20, or more than 20 PNA oligomers). A plurality of PNA oligomers can include, for example, one PNA oligomer targeting the transcription initiation site, a second PNA oligomer targeting the translation start site, and a third PNA oligomer targeting a sequence in between the transcription and translation start sites. Methods of the invention also are useful for treating multiple different mammals by administering to each mammal a plurality of PNA oligomers. The plurality of PNA oligomers can be designed as described herein (e.g., in Example 11), based on nucleotide sequence information from the mutant and non-mutant alleles from each of the multiple different mammals. A plurality of PNA oligomers can include, for example, PNA oligomers targeted to multiple sites within a single allele, wherein each of the multiple different sites is determined (e.g., by DNA sequencing) to be a potentially useful target for PNA therapy.

The invention provides methods of using PNA oligomers to elicit a certain biological response (e.g., a reduction in the level of an RNA or a polypeptide) in a sequence-specific manner. The biological response can be any alteration of a particular activity, such that the activity is increased, decreased, or abolished altogether. For example, the activity of an htt polypeptide encoded by an expanded HD allele can be decreased by a PNA oligomer targeted to the allele. Without being bound by a particular mode of action, a decrease in htt activity can be caused by, for example, a reduction in the level of htt polypeptide due to a PNA oligomer directed to the translational initiation site of an HD allele. For example, an anti-sense PNA oligomer can be used to target the translational start site and reduce the level of htt polypeptide expressed from a mutant HD mRNA. A decrease in htt activity also can be caused by a reduction in the level of mRNA encoding the polypeptide due to a PNA oligomer directed to the transcriptional start site. For example, a sense PNA oligomer can be used to target the transcription initiation site and reduce the level of htt mRNA expressed from a mutant HD allele.

Methods of the invention are useful for reducing the level of an RNA (e.g., an mRNA) or a polypeptide encoded by a mutant allele. The level of RNA or polypeptide encoded by a mutant allele typically is reduced to a greater extent than the reduction, if any, in the level of the RNA or polypeptide encoded by the non-mutant allele. As used herein, “reducing the level of an RNA or the level of a polypeptide” refers to any reduction (e.g., a 1% reduction, a 5% reduction, a 10% reduction, a 50% reduction, or a complete, 100% reduction) in the level of a particular RNA or polypeptide after administration of one or more PNA oligomers.

Similarly, methods of the invention are useful to reduce expression of a polypeptide from a mutant allele, typically to a greater extent than any reduction in expression from the non-mutant allele. The term “wherein expression of a polypeptide is reduced” refers to any reduction (e.g., a 1% reduction, a 5% reduction, a 10% reduction, a 50% reduction, or a complete, 100% reduction) in the level of a particular polypeptide after administration of one or more PNA oligomers.

RNA and polypeptide levels can be assessed using any of a number of methods, many of which are well known in the art. RNA levels can be measured using, for example, reverse transcription-PCR (RT-PCR), Northern blotting, or in situ hybridization. Levels of polypeptides can be measured by, for example, western blotting or enzyme-linked immunosorbance assay (ELISA). A reduction in the level of an RNA or a polypeptide expressed from a mutant allele that is associated with a particular disease also can be observed based on a reduction in disease symptoms or reversal of disease pathophysiology. A PNA oligomer directed against, for example, an expanded HD allele can be used to reduce expression from the mutant allele and reverse disease pathophysiology. Reversal of HD pathophysiology can be monitored by, for example, observing a reduction in HD symptoms (e.g., improved coordination and cognitive abilities) in a mammal such as a human. In an animal such as mouse, reversal of pathophysiology can be assessed using, for example, the methods described herein in Example 5.

The invention provides articles of manufacture that can include PNA oligomers combined with packaging material and can be sold as kits for reducing the pathophysiology of autosomal dominant diseases. Components and methods for producing articles of manufacture are well known. Articles of manufacture may combine one or more of the PNA oligomers provided herein. In addition, an article of manufacture further may include, for example, buffers or other control reagents for reducing or monitoring reduced expression from a mutant allele. The packaging materials can contain instructions describing how the PNA oligomers are effective for reducing expression of RNA and/or polypeptide from a mutant allele. The packaging materials also can contain instructions indicating which PNA oligomers should be administered to which type of patient. For example, instructions can indicate that a particular first PNA oligomer should be given to patients having a particular first mutant allele, while a particular second PNA oligomer should be given to patients having a particular second mutant allele 3, and so on.

The invention will be further described in the following examples, which do not limit the scope of the invention described in the claims.

### EXAMPLES

### Example 1

### Materials and Methods

Synthesis of PNA oligomers: PNA oligomers were synthesized by solid phase synthesis on 4-methylbenz-hydrylamine-HCl (MBHA) resin (Advanced ChemTech, Louisville, Ky.) using an N-[(di-methylamino)-1H-1,2,3-triazolo[4,5-b]pyridin-1-ylmethylene]-N-methylmethaminium hexafluorophosphate/N,N-diisopropylethylamine (HATU/DIPEA) activation mixture in N-methylpyrrodinilone (NMP) and the protected [2-[[(1,1-dimethylethoxy)carbonyl]amino]ethyl]-N-[2-[6-[[(phenylmethoxy) carbonyl] amino]-9H-purin-9-yl]acetyl]-glycine (Boc-A-monomer), N-[2-[1,6-dihydro-6-oxo-2-[[(phenylmethoxy)carbonyl]amino]-9H-purin-9-yl]acetyl]-N-[2-[[(1,1-dimethylethoxy)carbonyl]amino]ethyl]-glycine (Boc-G-monomer), N-[2-[[(1,1-dimethylethoxy)carbonyl]amino]ethyl]-N-[2-[2-oxo-4-[[(phenylmethoxy) carbonyl]amino]-1 (2H)-pyrimidinyl]acetyl]-glycine (Boc-C-monomer), and N-[2-[3,4-dihydro-5-methyl-2,4-dioxo-1 (2H)-pyrimidinyl]acetyl]-N-[2-[[(1,1-dimethylethoxy) carbonyl]amino]ethyl]-glycine (Boc-T-monomer).

After synthesis, the PNA molecules were deprotected and cleaved in a single operation by treating the resin with a solution containing 80% trifluoroacetic acid (TFA) and 20% m-Cresol for 90 minutes at 22° C. Crude PNA oligomers then were precipitated using cold anhydrous ether. The precipitated PNA oligomers were purified on a Vydac silica gel based column (C8, 22 mm×250 mm, 10 micron pore size, detection at 260 nm, flow rate 8 mL/min) with a buffer of 0.1% aqueous TFA and a linear gradient of 0.5% TFA containing 80% acetonitrile/20% water. The pooled fractions were lyophilized and stored as powders at −20° C.

Lyophilized PNA oligomers were dissolved in 10 μL of distilled water and heated to 80° C. for at least 5 minutes. The concentration of the dissolved PNA oligomers was determined by absorbance at 260 nm according to (OD₂₆₀×26×dilution)/1000=PNA concentration in μg/μL. Injection stocks were prepared by dilution into sterile physiological saline (0.9% NaCl) to a final concentration of 25 μg/μL. Aliquots of 1 mL were stored at −20° C. until use. PNA solutions were heated and cooled to room temperature prior to injection.

Cannulation and microsurgery—method 1: B6CBA, R6/1, and R6/2 mice were obtained from The Jackson Laboratory (Bar Harbor, Me.). Mice were anesthetized via intraperitoneal (i.p.) injection with 100 mg/kg of a ketamine/xylazine solution containing 8 mg/mL ketamine and 1 mg/mL xylazine in 0.9% saline. Once anesthetized, each mouse was cannulated. Each anesthetized mouse was placed in a Stoelting Stereotaxic Frame with a mouse adapter (Stoelting Instruments, Wood Dale, Ill.). An incision was made using a scalpel, and the skin and tissue were pulled back with swabs to reveal the skull. The cannulation site was positioned for optimum access to the ventricle, at coordinates AP −0.2 mm, Horiz −1 mm from bregma. A cannula entry port and two bone screw ports were opened in the skull using a Dremel Flex Shaft Drill (Stoelting Instruments) with a 2.1 mm burr for the guide cannula port and a 0.7 mm burr for the bone screws (Fine Science Tools, Foster City, Calif.). Two 4.0 mm long bone screws (0.85 diameter) were placed far enough into the bone screw ports to fasten securely. After placing the bone screws, a cannula system (Plastics One, Roanoke, Va.) including a guide cannula (C315Gs-5/Spc), a dummy cannula (C313Dcs-5Spc), an injection cannula (C3151S-5.2/Spc), and a connector assembly (C313C) was placed in the guide cannula port. The guide cannula was positioned 1.5 mm into the brain using a stereotaxic needle and was secured to the bone screws and incision with dental cement. Once the dental cement hardened, the stereotaxic needle was removed and both the injection cannula and dummy cannula were placed within the guide cannula and secured to the connector assembly. Proper cannula placement was confirmed in several mice by injecting bromophenol blue dye into the ventricular space and noting that only the ventricle was stained. Following surgery, the mice were allowed to recover under a heat lamp to maintain body temperature. Animals recuperated from surgery for five days prior to injection of PNA oligomers.

Cannulation and microsurgery—Method 2: Pump cannulas were prepared 48 hours before surgery. For adaptation to mice, Alzet pump cannulas were ground to a length of 2 mm. The cannulas then were sterilized by gas ozonation. Mice were anesthetized via i.p. injection with 100 mg/kg of a ketamine/xylazine solution containing 8 mg/mL ketamine and 1 mg/mL xylazine in 0.9% saline. Once anesthetized, each mouse was cannulated. Each anesthetized mouse was placed in a Stoelting Stereotaxic Frame with a mouse adapter (Stoelting Instruments). An incision was made using a scalpel, and the skin and tissue were pulled back with swabs to reveal the skull. The cannulation site was positioned for optimum access to the ventricle, at coordinates AP −0.2 mm, Horiz −1 mm from bregma. A cannula entry port and two bone screw ports were opened in the skull using a Dremel Flex Shaft Drill (Stoelting Instruments) with a 2.1 mm burr. The cannula was lowered into place and affixed using Loctite 454 (Loctite Corp., Avon, Ohio). An Alzet Mini-Osmotic Pump was inserted into a pouch beneath the skin and attached to the cannula with silastic tubing. The wound was closed using wound clips and the animal was allowed to recuperate.

Delivery of PNA oligomer solutions: All handling of tubing and cannulas was done under sterile conditions. PNA solutions at the correct concentrations were diluted in artificial cerebrospinal fluid (ACSF; 147 mM NaCl, 4.02 mM KCl, 1.2 mM CaCl₂, pH 7.5) and then filtered for delivery. As a control, ACSF containing no PNA was delivered.

Delivery of PNA oligomers was accomplished over a 1 minute time interval by slow injection into the cannulas, using a 10 μL Hamilton syringe connected to polyethylene (PE50) tubing. The injector was not removed from the cannula for at least 1 minute to prevent a vacuum effect. The dummy cannula replaced the guide cannula between treatments.

Animal treatment and tissue preparation: Aliquots of 1.0 μL (25 μg/μL) of a PNA oligomer specific for the expanded human HD allele (HDs) or a scrambled control PNA (HDscr) were administered into the ventricle every other day by injection via the cannula. During the treatment, animal behavior was normal, and no effects of toxicity as measured by weight or motor function relative to untreated animals were observed.

Following the treatment period, mice were anesthetized with ether and sacrificed by cervical dislocation. Whole brains and livers were removed and placed in 0.9% saline. Striatum tissue was dissected from whole brains, and liver tissue was finely minced. The tissues were placed in separate 2 mL microfuge tubes on dry ice and then suspended in 20 volumes of RIPA buffer (50 mM Tris pH 8, 150 mM NaCl, 1 mM EDTA, 1 mM EGTA, 0.5% sodium deoxycholate, 0.1% SDS, 1% TritonX-100, 2 μg/mL leupeptin, 2 μg/mL aprotinin, 1 μg/mL pepstatin, 1 mM phenylmethylsulfonylfluoride) per gram of tissue. Suspended tissues were homogenized by sonication, and the homogenates were clarified by centrifugation at 12,000×g for 20 minutes at 4° C. Following centrifugation, the supernatants were removed and protein concentrations were quantified by Bradford assays using a BSA standard.

Immunoblotting: Supernatants from the tissue homogenates were mixed with 1/6 volume of 6×SDS sample buffer (350 mM Tris pH 6.8, 10% SDS, 30% glycerol, 15% 2-mercaptoethanol, 0.012% bromophenol blue). Equal amounts of protein were directly loaded onto 10% acrylamide/0.05% bisacrylamide gels without heat denaturation. Proteins were electrophoretically separated and then transferred onto 0.22 μm nitrocellulose membranes in transfer buffer (25 mM Tris base, 192 mM glycine, 10% methanol, 0.025% SDS) at 12 V for 2 hours at room temperature (RT) using a submarine plate electrode unit (Idea Scientific, Minneapolis, Minn.). The membranes were air dried at RT and then blocked with 3% nonfat dry milk (NFDM) in TBS-T (50 mM Tris, pH 7.5; 150 mM NaCl, 0.05% Tween-20) for 1-2 hours at RT. The blocked membranes were washed 3 times for 5 minutes each in TBS-T and incubated overnight at 4° C. with primary antibodies diluted in antibody buffer (0.5% NFDM in TBS-T). A 1:1200 dilution of mAb 2166 (4C8; Chemicon Inc., Temecula, Calif.) was used to detect endogenous mouse htt, and a 1:3500 dilution of mAb 1574 (1C2; Chemicon Inc.) was used to detect human htt. Following incubation with the primary antibodies, membranes were washed in TBS-T. Membranes were incubated with secondary goat anti-mouse antibodies (33.3 ng/mL; Chemicon Inc.) for 1 hour at RT. Finally, the membranes were washed 4 times (5 minutes each wash) in TBS-T and 1 time for 5 minutes in TBS prior to addition of substrate (Pierce Super Signal West Pico; Pierce, Rockford, Ill.). Protein bands were detected on film by chemiluminescence.

Open field test to assess motility: Each mouse was confined for 10 minutes in a Plexiglas locomotion chamber equipped with a laser source and detector. As the mouse passed between the laser source and detector, the laser beam was “broken” and the event was automatically recorded. The number of beam breaks for B6CBA mice treated with the HDs PNA oligomer was compared to that of saline-treated or untreated control B6CBA mice. This number was directly related to general activity of the mice.

Clasping test: Dystonic movement was assessed using a procedure adapted from Yamamoto and colleagues (Yamamoto et al., Cell, 101:57-66, 2000). Each animal was suspended by the tail for 15 seconds, and its behavior was videotaped for subsequent analysis. The number and duration of abnormal movements were recorded upon viewing the videotaped behavior. An abnormal movement was defined as any dystonic movement of hindlimbs and/or forelimbs and/or trunk during which the limbs were pulled in toward the body in a manner distinct from that observed in wild-type mice. The duration of dystonic movements was recorded as a percent of total time. Mice were assessed for clasping score and clasping duration. The test period was divided into 2-second increments. Mice received a score of 1 if they displayed any abnormal movement during the given increment, allowing for a maximum score of 7.

Bar test to assess motor skill: Each mouse was held by its tail and allowed to grasp a bar (0.25 cm in diameter, 60 cm long) suspended 30 cm above a padded bench. After the mouse had firmly grasped the bar, the test was begun by releasing its tail. The mouse was allowed two minutes to “escape” by climbing along the bar to one of the bar supports. The mouse was given both performance and time ratings. Performance ratings were based on the following scale: 0, unable to hold bar; 1, holds bar, unable to bring hind limbs to bar; 2, holds bar, draws hind limbs to bar (i.e., 3 paws firmly on bar); 3, holds bar, moves along bar (i.e., moves at least 2 inches in a coordinated manner along bar); 4, escapes (i.e., mouse touches a bar support with one paw). Time ratings were based on the following scale: 0, unable to hold bar (0-3 seconds); 1, holds bar for 4-30 seconds; 2, holds bar for 31-60 seconds; 3, holds bar for 61-90 seconds; 4, holds bar for 91-120 seconds. If the mouse had not reached one of the bar supports by the end of the two minute test period it was removed from the bar. The two ratings for each mouse were combined to produce a performance-time rating for that mouse. The performance-time ratings were assigned scores (see Table 1). Each mouse was tested three times, and the assigned scores were averaged for each mouse. The resulting averages were averaged within each group to represent the motor skill of that group.

TABLE 1
Bar Test Scoring System

| Performance-Time Rating | Assigned Score |
|---|---|
| 4-1 | 16 |
| 4-2 | 15 |
| 4-3 | 14 |
| 4-4 | 13 |
| 3-1 | 12 |
| 3-2 | 11 |
| 3-3 | 10 |
| 3-4 | 9 |
| 2-4 | 8 |
| 2-3 | 7 |
| 2-2 | 6 |
| 2-1 | 5 |
| 1-4 | 4 |
| 1-3 | 3 |
| 1-2 | 2 |
| 1-1 | 1 |

Rotating rod test: Coordination and balance are assessed using a procedure adapted from Carter and colleagues (Carter et al., J. Neurosci., 19:3248-3257, 1999). A stable baseline is established by training the mice to stay positioned on the rotating rod at a consistent speed (24 rpm) for a maximum of 60 seconds. Training takes place for 3 days with 4 trials per day. On day 4, the ability of the mice to remain on the rotating rod is assessed at 5, 10, 15, 20, 25, and 30 rpm.

Evaluation of brains after PNA treatment: To detect inclusions, fresh frozen coronal sections were obtained from brains isolated from treated mice. Mice were anesthetized and subjected to transcardial perfusion with 4% paraformaldehyde in physiological saline. Brains were removed, post-fixed for 1 hour at 4° C., and cryoprotected overnight in a 30% sucrose solution in 0.1 M PBS. 20 μm sections were cut on a cryostat and thaw-mounted onto slides. Immunohistochemistry was performed on both fresh frozen and fixed sections. An anti-Ubiquitin antibody was used at 1:500, and 2166 antibodies were used at 1:2000. Sections stained with 2166 were counterstained with cresyl violet.

To evaluate size of brain structures, a series of measurements (anterior to posterior) were taken on matched sections of each brain. Three matched sections were used for striatal area measurements. Measurements were taken in units of square micrometers by Stereoimager and converted to percent control. Successive measures were analyzed by repeated-measures ANOVA followed by Fisher post-hoc tests.

Creatine measurement: A non-invasive nuclear magnetic resonance (NMR) spectroscopy-based method also is used to monitor the response to PNA treatment. Creatine is a precursor for intracellular ATP levels and is easily detected by [¹H]-NMR methodology in whole animals. During long-term treatment, levels of creatine are measured and correlated to cell viability and animal health without sacrificing the animal. [¹H]-NMR in vivo spectroscopy is performed at 7 Tesla using an Avance DRX 300 NMR instrument equipped with mini and microimaging accessories (Bruker Instruments, Billerica Mass.). Mice are anesthetized using halothane/O₂/N₂O anesthesia (1.5% halothane; 2:1 O₂/N₂O). Body temperature is maintained using a stream of warm air at 38° C. Once anesthetized, each mouse is subjected to localized proton spectroscopy using either a PRESS sequence (see Bottomley, Ann. NY Acad. Sci., 508:333-348, 1987) with an echo time of 100-150 ms and a repetition time of 2 seconds, or a STEAM sequence (see Frahm et al., J. Magn. Reson., 72:502-508, 1987) with an echo time of 40 ms or less. Spectral width is 2 kHz with 1024 complex points. The transmitter frequency is set between the N-acetyaspartate (NAA) and creatine resonances. During data collection, voxel position and size is optimized to obtain the best signal-to-noise ratio and spatial selectivity. After data collection, the resulting spectra are analyzed using the XWIN software program (Bruker Instruments) and the Magnetic Resonance User Interface (MRUI) web site available on the internet. The NAA and total creatine values from the analyzed spectra are used to generate a separate ratio with the choline peak obtained from time domain fitting of the acquired signal. Creatine signals are monitored once a month during the treatment procedure. If sense PNA oligomers reverse HD pathophysiology, the levels of creatine typically improve in the brains of PNA-treated animals relative to untreated or saline treated controls.

### Example 2

### Design and Synthesis of Sense PNA Oligomers

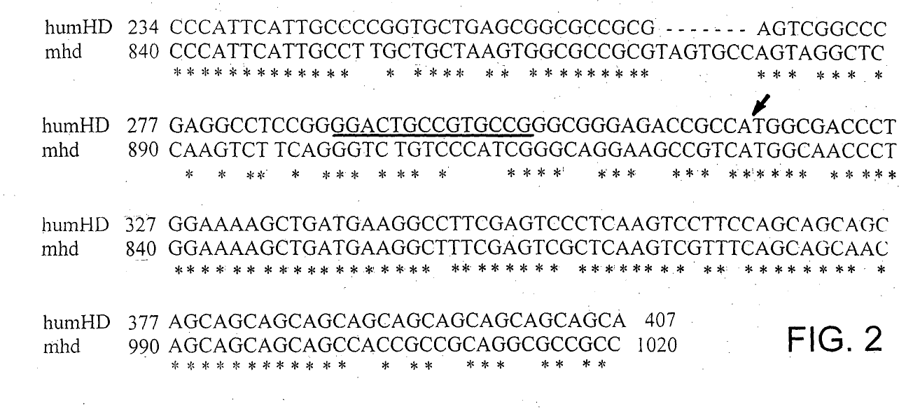

R6/1 and R6/2 mice harbor human HD transgenes that differ in the number of CAG repeats contained in exon 1: the R6/1 transgene contains 114 repeats, while the R6/2 transgene has 145 repeats. Both strains have a single integrated HD transgene and retain an endogenous mouse Hdh gene. The mouse and human HD genes from a B6CBA (parental strain of the R6/2 line) transgenic mouse were sequenced. The sequences were analyzed to identify a target region close to the translational start sites where the human and mouse sequences maximally diverge (see FIG. 2). The analysis revealed a target region 14 nucleotides in length with its 5′ end beginning at nucleotide −28 from the adenine in the start codon of the HD sequence. The identified target region has the following sequence: 5′-GGACTGCCGTGCCG-3′ (SEQ ID NO:3). This target region sequence corresponds to nucleotides 288-301 of the HD mRNA (GenBank Accession # L12392). A sense PNA (HDs) complementary to the identified target region sequence and a PNA oligomer with a scrambled target region sequence (HDscr; 5′-GCAGCGGCGGTCCT-3′; SEQ ID NO:4) were synthesized as described in Example 1.

### Example 3

### Selective Inhibition of the Expanded Human HD Allele in R6/1 Mice

R6/1 mice were divided into groups designated to receive either HDs or HDscr. Mice were cannulated as described above, and 1 μL aliquots (25 μg/μL) of HDs or HDscr were administered into the ventricle every other day for 4 or 6 days by injection via the cannula. Animals were sacrificed after treatment, and liver and brain tissues were prepared for immunoblotting.

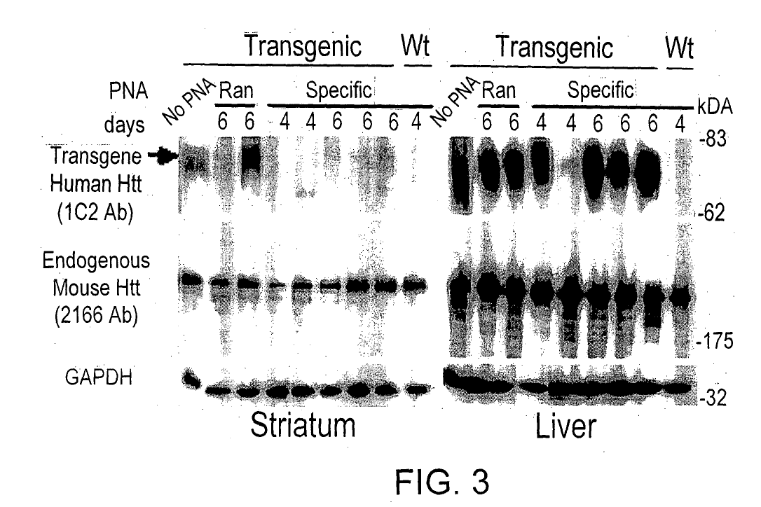

Mice treated with HDs for 4 or 6 days exhibited a dramatic inhibition of human HD transgene expression as compared to untreated R6/1 control animals (FIG. 3). The HDs PNA oligomer was significantly more effective at inhibiting transgene expression than was the random, scrambled PNA (shown as “Ran” in FIG. 3). Direct injection of HDs into the brain had little effect on expression of the human transgene in the liver. These results indicate that the systemic concentration of PNA after direct brain injection did not reach a level high enough to effectively inhibit human HD gene expression in peripheral tissues within the time interval tested. Neither HDs nor HDscr had any effect on expression of the endogenous mouse Hdh allele in any tissue examined at any of the tested time points. No measurable inhibition of non-targeted genes such as GAPDH was observed in any animal tested. These data demonstrate that inhibitory PNA oligomers can be designed to inhibit the disease allele without affecting expression of the normal endogenous mouse allele.

### Example 4

### Reversing Pathophysiology of Huntington Disease Using PNA Oligomers Targeting Human HD

R6/2 rather than R6/1 mice were used in these experiments. The transgene in R6/2 mice has a longer CAG repeat, resulting in the development of a more severe disease phenotype and an earlier age of onset. Although R6/2 mice develop normally, they show significant brain atrophy and loss of body weight relative to normal litter mates between 5-7 weeks of age. By week nine, concomitant with brain atrophy, R6/2 mice develop features of movement disorders such as irregular gait, tremors, and epileptic seizures.

Experiments were conducted to determine whether PNA-mediated inhibition of mutant huntingtin expression increased survival and/or reversed features of pathophysiology. R6/2 and non-transgenic mice were divided into two groups of three to five mice each, and treated with either HDs or ACSF for approximately ten weeks, beginning at five to six weeks of age. Treated animals received a dose of 0.15 mg/kg of the HDs PNA oligomer, administered by continuous infusion through an Alzet pump. All ACSF-treated transgenic mice died by three months of age. In contrast, the three animals that were treated with the HDs PNA oligomer had improved survival. One of the animals was still alive and healthy at six months of age.

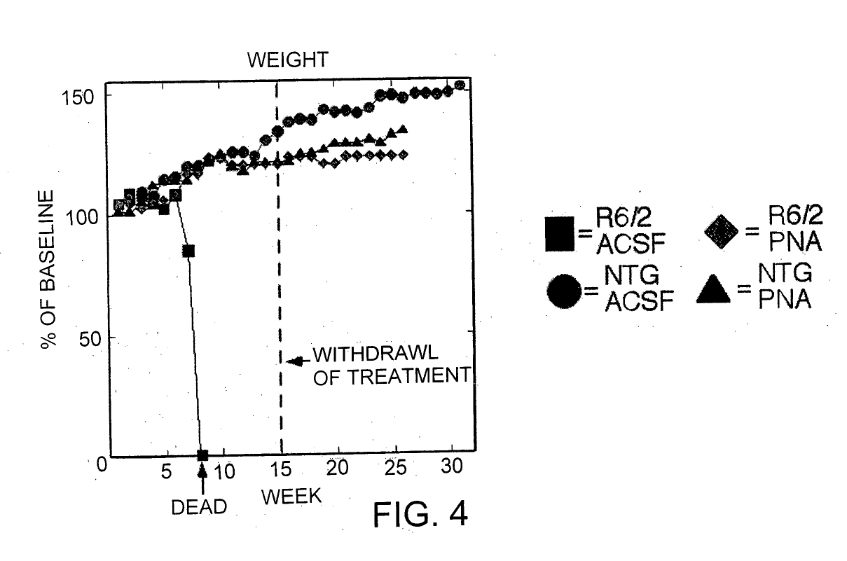

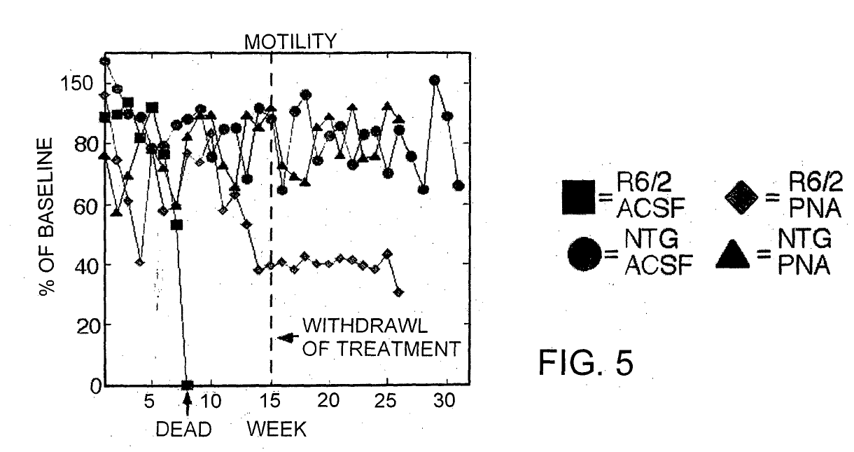

Quantification of the weights (FIG. 4) and motility (FIG. 5) of the surviving animals revealed that the PNA-treated transgenic mice continued to gain weight over time but did experience a decline in motility as compared to the non-transgenic animals. These data indicate that survival was not accompanied by a complete reversal of the pathophysiology. Examination of the two treated R6/2 mice that did not survive revealed that their pumps were partially clogged by precipitation of the PNA. Impaired delivery of the PNA oligomer thus may have accounted for the difference in survival time within the group of genetically identical animals. Despite their eventual death, the two non-surviving, PNA-treated R6/2 mice did exhibit improved pathophysiology and increased brain weight. Thus, PNA treated animals did not display the general brain atrophy that is typical of untreated R6/2 mice. All non-transgenic animals appeared unaffected by treatment with the HDs PNA oligomer.

In other experiments, R6/2 mice were divided into five groups (designated groups 1-5) containing eight to ten animals each. Groups 1-4 received either saline (group 1) or a specific amount of HDs (groups 2-4). Group 5 included untreated R6/2 mice. A sixth group included untreated non-transgenic B6CBA control mice. At 8 weeks of age, each mouse was weighed to establish a weight stability baseline. Baselines for clasping behavior, motility, and motor skill were established at 9 weeks of age using the methods described in Example 1.

At 9 weeks of age, the mice in each group received saline (group 1) or HDs at 2.0 mg/kg, 5.0 mg/kg, or 20.0 mg/kg (groups 2, 3, and 4, respectively) by i.p. injection every 48 hours. The injections continued through 13 weeks of age (i.e., treatment for five weeks). Weight stability, clasping behavior, motility, and motor skill were assessed weekly during the treatment period. At the time of death or after the treatment period, the brain of each mouse was removed using standard methods. The brains were divided into hemispheres, and one hemisphere of each brain was saved for protein and mRNA analysis. The remaining hemispheres were fixed directly in 10% formalin for 5-7 days. After fixing, the brain hemispheres were weighed individually and processed for sectioning and pathophysiology.

At 13 weeks of age, all non-transgenic mice (group 6) were alive and all untreated R6/2 mice (group 5) were dead. In addition, 50% of the mice treated with 5.0 mg/kg HDs and 20% of the mice treated with 20.0 mg/kg HDs were alive. None of the mice treated with 2.0 mg/kg HDs were alive at 13 weeks of age.

Evaluation of weight data revealed that mice treated with 5.0 mg/kg HDs exhibited more stable weight over the treatment period when compared to mice treated with either 2.0 mg/kg or 20.0 mg/kg HDs. Untreated non-transgenic mice gained weight and untreated R6/2 mice lost weight over the treatment period.

An open field test was used to assess the motility of the treated, untreated, and control mice. Analysis of the motility data revealed that mice treated with either 5.0 mg/kg or 20.0 mg/kg HDs displayed levels of motility that were increased as compared to untreated R6/2 mice, but lower than the level of motility displayed by nontransgenic control mice. Animals treated with 2.0 mg/kg HDs exhibited no difference in motility when compared to untreated R6/2 mice.

A bar test was used to evaluate motor skill in treated and untreated animals. Mice treated with either 5.0 mg/kg or 20.0 mg/kg HDs exhibited increased motor skill when compared to untreated or saline treated R6/2 mice. No significant alterations in clasping behavior were observed in any group over the treatment period.

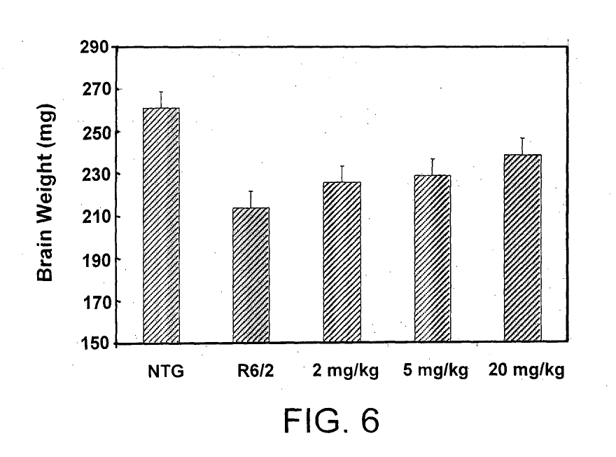

Treatment with HDs revealed a dose dependent effect on brain hemisphere weight. The brain hemisphere weights for all doses of PNA oligomers were greater than the weights of brain hemispheres from untreated R6/2 mice (FIG. 6). Mice treated with 20.0 mg/kg HDs exhibited the greatest degree of recovery in brain hemisphere weight as compared to untreated non-transgenic mice.

These data demonstrate that sense PNA oligomers having specificity for HD can improve the weight stability, motility, and motor skill of mice having late-stage Huntington disease. These data also reveal that PNA oligomers administered by i.p. injection were effective for treating a condition originating in the brains of these animals.

### Example 5

### Determination of the Minimum Dose of a Sense PNA that Effectively Inhibits Expression from the Human HD Transgene

45 R6/1 mice are randomly separated into 9 groups of 5 mice each. Groups 1-4 are designated sense PNA groups, and groups 5-8 are designated scrambled PNA groups. Group 9 is designated the control group. The mice are anesthetized and cannulated as described in Example 2. Cannulated mice are treated for 2, 3, 4, 5, or 6 days with 1 μg, 5 μg, 10 μg, or 25 μg sense PNA oligomers specific for the HD allele (groups 1-4, respectively), 1 μg, 5 μg, 10 μg, or 25 μg scrambled control PNA oligomers (groups 5-8, respectively), or physiological saline vehicle alone (group 9). PNA oligomers and vehicle alone are administered in 1 μL aliquots into the ventricle via the injection cannula. PNA oligomers or vehicle alone are delivered slowly over 1 minute using a 10 μL Hamilton syringe connected to PE50 tubing. After delivery, the syringe is left in the cannula for at least 1 minute to prevent a vacuum effect. The dummy cannula replaces the guide cannula between treatments. Mice treated for 6 days are administered PNA oligomers the day following the recovery period, while the rest of the mice in that group receive vehicle alone. Mice treated for 5 days are administered PNA oligomers starting two days after the recovery period, while the rest of the mice in that group receive vehicle alone. Similar treatments are carried out with mice treated for 4, 3, and 2 days. Following the treatment period, mice are anesthetized with ether and sacrificed by cervical dislocation.

Whole brains are removed, placed in 0.9% saline, and separated into hemispheres. Hippocampus, neostriatum (caudate and putamen), cerebral cortex, and cerebellum tissues are dissected and placed in 2 mL microfuge tubes on dry ice. The dissected tissues are suspended in 20 volumes of RIPA buffer per gram of tissue, and suspended tissues are homogenized by sonication. The resulting homogenates are clarified by centrifugation, assayed for protein concentration, separated by gel electrophoresis, transferred to nitrocellulose membranes, and processed as described above to detect expression products from the human HD transgene or the endogenous mouse Hdh gene. The effect of PNA administration on human and mouse htt polypeptide levels is then evaluated.

Selective inhibition of polypeptide expression is taken as a reduction in intensity of the human htt band (between 62 and 83 kD) with retention of the full-length mouse htt band above 175 kD. The half-life of the htt polypeptides is estimated to be 24-36 hours (see Persichetti et al., Neurobiol. Dis., 3:183-190, 1996). An observation time of six days represents at least three half lives and typically is sufficient to detect selective changes in htt polypeptide levels.

### Example 6

### Determination of the Longest Interval Between Doses of Sense PNA Oligomers that Results in Selective Inhibition of Expression from the Human HD Transgene

R6/2 or control B6CBA mice are divided into experimental groups. Each group is subjected to a different injection interval or a different PNA oligomer, or serves as a control. R6/2 rather than R6/1 mice are used in these and all subsequent experiments, for the reasons stated above (see Example 4).

Cannulated mice are divided into 15 experimental groups. Groups 1-4 contain R6/2 transgenic mice that receive sense PNA oligomers every other day (group 1), every week (group 2), every two weeks (group 3), or every month (group 4). These mice are used to evaluate the effect of injection interval on maintaining reduced polypeptide expression. R6/2 groups 5-8 receive randomized PNA oligomers as a control in parallel with groups 1-4 at the same injection intervals. Group 9 mice receive vehicle alone every other day. Groups 10 and 11 contain untreated R6/2 mice and untreated B6CBA control mice, respectively.

The effect of PNA treatment on reducing htt expression in R6/2 mice is evaluated as a function of injection interval. Expression from the HD transgene and the endogenous mouse Hdh gene is evaluated as described above. Inhibition of transgene expression is evaluated by comparing levels of htt expression in mice treated with either sense PNA, random scrambled PNA, or saline with the level of htt expression in untreated R6/2 mice (group 10) and wild type B6CBA mice (group 11). R6/2 mice typically exhibit a full range of expression of both the human transgene and the mouse allele, while B6CBA mice have no expression of the human transgene. Inhibition of human htt expression is calculated as the ratio of the average level of transgene expression in PNA-treated mice to that of the untreated controls. The intensities of the transgene bands from groups 1-4 and group 10 observed using phosphoimager analysis is used to estimate the average fractional reduction in transgene expression relative to that average in untreated R6/2 mice. Similarly, the maintenance of endogenous mouse htt expression is quantified using band intensities observed using phosphoimager analysis. The optimum injection interval is taken as the longest time between injections that maintains reduced human HD transgene expression without reducing expression of the endogenous mouse Hdh gene. In other words, if selective inhibition can be maintained using injections every month, injections will be given every month rather than, for example, every other day.

The toxicity of HDs PNA oligomers over different intervals of administration is evaluated in B6CBA control mice that do not harbor the human transgene, i.e., groups 11-15. Mice in group 11 are untreated B6CBA mice. Mice in groups 12-15 receive HDs PNA oligomer injections every other day (group 12), every week (group 13), every two weeks (group 14), and every month (group 15). Toxicity is evaluated in B6CBA animals by weight loss, coat appearance, decrease in locomotion or, if severe adverse effects occur, a decrease in survival time induced by PNA oligomer treatment.

The weight, coat appearance, and general activity level of the mice in each group is monitored and recorded on a weekly basis. The weight of each mouse is recorded at the beginning of the study and is monitored and recorded every week thereafter. If changes in the animal weight become pronounced, the frequency of measurement is increased to every other day. A second measure of toxicity is coat appearance. Mice normally have shiny, smooth coats that are groomed regularly. If mice develop toxic reactions, grooming is diminished, and the coat appears coarse or patchy. At the time of weighing, coat appearance also is observed and recorded. General activity serves as a third measure of toxic side-effects. The general activity of each mouse is evaluated using an open field test. If PNA treatment produces no toxic effects, then the number of beam breaks will not be significantly different among the groups.

### Example 7

### Determination of the Specificity of a PNA Oligomer Using Global Chip-Based mRNA Analysis

Gene expression is measured using the Affymetrix Murine Genome U7 set comprising three arrays representing about 36,000 full-length genes and EST clusters (Affymetrix, Santa Clara, Calif.). The first array in the set (MG-U74A) represents about 6000 functionally characterized sequences in the Mouse UniGene database, as well as about 6000 EST clusters. The arrays are designed so that each gene is represented by multiple (about 20) oligonucleotide probe pairs biased toward the 3′ end of the gene. To help identify non-specific and background signal, each oligonucleotide probe pair consists of a perfect match oligonucleotide and a mismatch oligonucleotide identical in sequence to the perfect match oligonucleotide except for a homomismatch at the center base. Arrays also contain multiple reference genes so that standards can be added to a sample prior to hybridization, facilitating normalization and quantification. Experimental procedures and analyses are carried out according to the manufacture's specifications.

Trizol (Invitrogen/Life Technologies, Carlsbad, Calif.) is used to extract total RNA from the striata of mice treated with sense PNA oligomers or from untreated control mice. Once extracted, the polyadenylated RNA is purified on oligo-dT-linked Oligotex resin (Qiagen, Valencia, Calif.). Double-stranded cDNA containing a T7 RNA polymerase promoter is prepared from the purified polyadenylated RNA using SuperScript reverse transcriptase (Invitrogen/Life Technologies). The resulting double-stranded cDNA is used to prepare biotinylated cRNA by transcription using T7 Megascript (Ambion, Inc., Austin, Tex.). The biotinylated cRNA is then hybridized to the arrays. Following hybridization, the arrays are washed to remove non-specifically bound cRNA. The bound biotinylated cRNA is stained with a streptavidin-phycoerythrin conjugate. Fluorescence intensity data are captured using an array scanner, and intensity data are calculated and averaged for each probe cell. The average intensity for each gene, which correlates with mRNA abundance, is calculated from the probe cell intensities. The number of events in which the perfectly matched hybridization signal is larger than the mismatched signal is computed along with the average of the log of the perfect/mismatch ratio. Data are sorted by an analysis parameter and are used to evaluate gene clusters that are enhanced or inhibited by sense PNA treatment.

### Example 8

### Determining when Treatment with PNA Oligomers Most Effectively Reverses Disease Pathophysiology

Three groups containing 6 mice each are included in this study: two groups of R6/2 mice (groups 1 and 2) and one group of wild type B6CBA mice (group 3). All mice are cannulated at 5 weeks of age. Group 1 receives HDs at a dose and interval as determined in Examples 3 and 4. Groups 2 (R6/2 mice) and 3 (control B6CBA mice) receive saline over the same interval. A successful PNA treatment is defined as improvement of the group 1 mice (e.g., improved weight gain, coat appearance, and general activity) while the group 2 mice undergo disease progression similar to untreated R6/2 mice. The B6CBA mice serve as normal wild type controls.

The efficacy of PNA treatment in reversing pathophysiology in R6/2 animals is evaluated at 3, 10, 15, 20 and 24 weeks, or as long as the animals survive. The degree to which sense PNA treatment can reverse pathophysiology is evaluated by comparing the phenotype of mice in group 1 with those of the mice in groups 2 and 3. The ability of sense HD PNA oligomers to reverse pathophysiology is assessed by monitoring weight alterations and coat appearance (as described in Example 5), recovery of motor skill, and survival.

Recovery of motor skill is measured by assessing dystonic movement or coordination and balance. One of the earliest manifestations of the motor phenotype is a dyskinesia of the limbs, which is displayed as a clasping of the limbs toward the body when an affected mouse is suspended by the tail. This phenotype typically is apparent by 4 weeks of age and becomes increasingly worse as the mouse ages. Mice are assessed every 2 weeks after 3 weeks of age.

In addition, locomotor abnormalities are evaluated in R6/2 mice starting at 5 weeks of age, when coordination and balance are measured using a rotating rod. Mice between 3 and 52 weeks of age are evaluated every 2 weeks.

Mice are sacrificed at the end of treatment, and the levels of mRNA or polypeptide expression from the human HD transgene and the endogenous mouse Hdh gene are measured to determine if selective inhibition of transgene expression has occurred. If expression of the transgene is inhibited by administration of the HDs PNA, the appearance of the behavioral phenotype typically is prevented or attenuated to the degree that the transgene expression is inhibited. Reversal of the disease pathophysiology is exhibited by the maintenance of weight and coat appearance, reversal of the clasping phenotype, and improved coordination and balance in a PNA oligomer-treated mouse.

### Example 9

### Evaluation of the Best Route of Administration for HDs PNA

Two routes of administration are evaluated and compared: i.p. injection and oral administration via drinking water. The optimal HDs dose and treatment interval for both routes of administration are determined as described in Examples 5 and 6. For both i.p. injection and drinking water studies, R6/2 mice are divided into three groups: group 1 receives HDs, group 2 receives HDscr, and group 3 receives vehicle alone injected directly into the i.p. cavity. The effect on transgene expression is evaluated at 3 months, 6 months, and one year as described in Examples 2, 5, and 6. If sense PNA treatment reverses pathophysiology, then weight gain, reappearance of coat quality, and improvement of motor function are observed. In addition, a non-invasive NMR spectroscopy-based method is used to monitor the response to PNA treatment.

At 3 months, 6 months, and one year after injections begin, mice are sacrificed and the brains are removed. The levels of polypeptide and mRNA expressed from the endogenous mouse Hdh gene and the human HD transgene are analyzed as described above. The experiments are repeated with alterations in the amount of i.p. injected PNA oligomers as needed. Untreated R6/2 mice typically undergo a characteristic disease progression leading to death.

### Example 10

### PNA Design Based on HD Allele Sequences from Individuals with HD

To test the ability of PNA oligomers to discriminate between sequences with unique or few nucleotide differences, the effect of PNA oligomers on disease and normal human alleles is measured. In order for a PNA oligomer to inhibit an HD-associated HD allele, there must be one or more single nucleotide polymorphisms (SNPs) that distinguish the mutant allele from the normal allele. The most useful sequence differences often reside in nucleotide sequences that flank the coding sequence of the mutant gene, including the translation start site. Sequence differences within the coding sequence, however, can be used.

To identify target regions, both the normal and the disease-associated HD alleles are sequenced. For example, PCR primer sets are designed to generate multiple amplicons of about 200 bases. These amplicons can cover the HD promoter and 5′ regulatory sequences. Sequencing primers that are internal to the amplicons also are generated.

The sequences are aligned using computer alignment software, and sequence variations (e.g., SNPs) are identified. Criteria for target site selection include (1) sequences close to the translational start site or other key regulatory regions, and (2) sites in which the normal and mutant sequences maximally diverge. One or more PNA oligomers are synthesized that are complementary to the mutant sequence at sites that are most divergent from the normal sequence. PNA oligomers typically are 14-20 residues in length. For each individual PNA, a control PNA with a random sequence also is synthesized. To target multiple sequences simultaneously, a “cocktail” of PNA oligomers is prepared.

The PNA oligomers are administered to cultured cells or to animals to evaluate their ability to mediate selective inhibition. PNA oligomers are added to the culture medium or are administered to animals, and expression of the normal and mutant htt polypeptides is evaluated over time. Polypeptide expression is detected by western blotting using the 1C2 antibody, which recognizes mutant htt containing an expanded glutamine tract, and the 2166 antibody, which detects both mutant and normal htt.

One or more unique PNA oligomers typically are designed for each affected individual, although the same PNA oligomers can be used to treat multiple affected family members if similar alleles are inherited. It also is possible that therapeutic PNA sequences targeted to a particular disease gene are limited such that one of several sizes fits all. Treatment of an affected individual with more than one PNA oligomer allows multiple sites within the mutant allele to be targeted.

### Example 11

### Treating Humans with PNA Oligomers

Sequence comparison as described in Example 11 is used to design PNA oligomers for treating humans having a particular disorder (e.g., HD). A plurality of PNA oligomers are synthesized based on the sequences of the wild type and mutant alleles from an individual. For example, the HD alleles from a first individual are sequenced, revealing a number of potential target sites. Those sites that are within the promoter region and exhibit the most differences between the two alleles are selected, and PNA oligomers directed toward those sites are synthesized. This plurality of PNA oligomers then is administered to the first individual. PNA oligomers are administered individually or as a “cocktail.” The procedure is repeated for a second individual even though the first individual and the second individual have the same disorder. The second individual therefore is treated with a plurality of PNA oligomers that are designed based specifically on the sequences of his or her alleles. The success of the treatment is determined by monitoring disease symptoms and observing whether there is a reversal in disease pathophysiology.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing the level of an RNA or the level of a polypeptide in a mammal having a mutant allele that causes a dominant disorder, wherein said RNA and said polypeptide are encoded by said mutant allele, and wherein said mammal is heterozygous for said mutant allele, said method comprising administering a polyamide nucleic acid oligomer to said mammal under conditions wherein the level of said RNA is reduced to a greater extent than the reduction, if any, in the amount of a second RNA or the level of said polypeptide is reduced to a greater extent than the reduction, if any, in the amount of a second polypeptide, wherein said second RNA and said second polypeptide are encoded by a second allele in said mammal, and wherein said second allele corresponds to said mutant allele and does not cause said dominant disorder.

2. The method of claim 1, wherein said mammal is a human.

3. The method of claim 1, wherein said dominant disorder is an autosomal dominant disorder.

4. The method of claim 1, wherein said dominant disorder is Huntington disease.

5. The method of claim 1, wherein said RNA is mRNA.

6. The method of claim 1, wherein said polyamide nucleic acid oligomer is administered into the brain of said mammal.

7. The method of claim 1, wherein said polyamide nucleic acid oligomer is administered intraperitoneally to said mammal.

8. The method of claim 1, wherein said polyamide nucleic acid oligomer comprises a sequence having specificity for a transcription initiation site of said mutant allele, a translation initiation site of said mutant allele, or a region between said transcription initiation site and said translation initiation site of said mutant allele.

9. The method of claim 1, wherein said polyamide nucleic acid oligomer comprises the sequence set forth in SEQ ID NO:3.

10. A method for treating a dominant disorder caused by a mutant allele in a mammal, said method comprising:
a) obtaining a polyamide nucleic acid oligomer based on sequence information obtained from said mammal, wherein said polyamide nucleic acid oligomer has specificity for said mutant allele; and
b) administering said polyamide nucleic acid oligomer to said mammal under conditions wherein expression of a first polypeptide is reduced, wherein the amount of reduction in expression of said first polypeptide is greater than the amount of reduction, if any, in expression of a second polypeptide, wherein said first polypeptide is encoded by said mutant allele, wherein said second polypeptide is encoded by a second allele in said mammal, and wherein said second allele corresponds to said mutant allele and does not cause said dominant disorder.

11. The method of claim 10, wherein said mammal is a human.

12. The method of claim 10, wherein said dominant disorder is an autosomal dominant disorder.

13. The method of claim 10, wherein said dominant disorder is Huntington disease.

14. The method of claim 10, wherein said sequence information was obtained by PCR.

15. The method of claim 10, wherein said polyamide nucleic acid oligomer is administered into the brain of said mammal.

16. The method of claim 10, wherein said polyamide nucleic acid oligomer is administered intraperitoneally to said mammal.

17. The method of claim 10, wherein said polyamide nucleic acid oligomer comprises a sequence having specificity for a transcription initiation site of said mutant allele, a translation initiation site of said mutant allele, or a region between said transcription initiation site and said translation initiation site of said mutant allele.

18. The method of claim 10, wherein said polyamide nucleic acid oligomer comprises the sequence set forth in SEQ ID NO:3.

19. A method for treating a dominant disorder caused by a mutant allele in a mammal, said method comprising:
a) obtaining at least a portion of the sequence of said mutant allele;
b) obtaining a polyamide nucleic acid oligomer based on said sequence, wherein said polyamide nucleic acid oligomer has specificity for said mutant allele; and
c) administering said polyamide nucleic acid oligomer to said mammal under conditions wherein expression of a first polypeptide is reduced, wherein the amount of reduction in expression of said first polypeptide is greater than the amount of reduction, if any, in expression of a second polypeptide, wherein said first polypeptide is encoded by said mutant allele, wherein said second polypeptide is encoded by a second allele in said mammal, and wherein said second allele corresponds to said mutant allele and does not cause said dominant disorder.

20. The method of claim 19, wherein said mammal is a human.

21. The method of claim 19, wherein said dominant disorder is an autosomal dominant disorder.

22. The method of claim 19, wherein said dominant disorder is Huntington disease.

23. The method of claim 19, wherein said sequence was obtained by PCR.

24. The method of claim 19, wherein said polyamide nucleic acid oligomer is administered into the brain of said mammal.

25. The method of claim 19, wherein said polyamide nucleic acid oligomer is administered intraperitoneally to said mammal.

26. The method of claim 19, wherein said polyamide nucleic acid oligomer comprises a sequence having specificity for a transcription initiation site of said mutant allele, a translation initiation site of said mutant allele, or a region between said transcription initiation site and said translation initiation site of said mutant allele.

27. The method of claim 19, wherein said polyamide nucleic acid oligomer comprises the sequence set forth in SEQ ID NO:3.

28. A method of assisting a medical professional in treating a dominant disorder caused by a mutant allele in a mammal, said method comprising providing a polyamide nucleic acid oligomer based on sequence information obtained from said mammal, wherein said polyamide nucleic acid oligomer has specificity for said mutant allele, wherein administration of said polyamide nucleic acid oligomer reduces expression of a first polypeptide in said mammal, wherein the amount of reduction in expression of said first polypeptide is greater than the amount of reduction, if any, in expression of a second polypeptide, wherein said first polypeptide is encoded by said mutant allele, wherein said second polypeptide is encoded by a second allele in said mammal, and wherein said second allele corresponds to said mutant allele and does not cause said dominant disorder.

29. The method of claim 28, wherein said mammal is a human.

30. The method of claim 28, wherein said dominant disorder is an autosomal dominant disorder.

31. The method of claim 28, wherein said dominant disorder is Huntington disease.

32. The method of claim 28, wherein said sequence information was obtained by PCR.

33. The method of claim 28, wherein said polyamide nucleic acid oligomer comprises a sequence having specificity for a transcription initiation site of said mutant allele, a translation initiation site of said mutant allele, or a region between said transcription initiation site and said translation initiation site of said mutant allele.

34. The method of claim 28, wherein said polyamide nucleic acid oligomer comprises the sequence set forth in SEQ ID NO:3.

35. A method of assisting a medical professional in treating multiple different mammals, wherein each of said multiple different mammals has a dominant disorder caused by a mutant allele, said method comprising providing a plurality of different polyamide nucleic acid oligomers based on sequence information obtained from each of said multiple different mammals, wherein at least one of said plurality of different polyamide nucleic acid oligomers has specificity for said mutant allele from each of said multiple different mammals such that administration of said at least one of said plurality of different polyamide nucleic acid oligomers to each of said multiple different mammals reduces expression of a first polypeptide in each of said multiple different mammals, wherein the amount of reduction in expression of said first polypeptide is greater than the amount of reduction, if any, in expression of a second polypeptide, wherein said first polypeptide is encoded by said mutant allele, wherein said second polypeptide is encoded by a second allele in each of said multiple different mammals, and wherein said second allele corresponds to said mutant allele and does not cause said dominant disorder.

36. The method of claim 35, wherein each of said multiple different mammals is a human.

37. The method of claim 35, wherein said dominant disorder is an autosomal dominant disorder.

38. The method of claim 35, wherein said dominant disorder is Huntington disease.

39. The method of claim 35, wherein said sequence information was obtained by PCR.

40. The method of claim 35, wherein each of said plurality of polyamide nucleic acid oligomers comprises a sequence having specificity for a transcription initiation site of said mutant allele, a translation initiation site of said mutant allele, or a region between said transcription initiation site and said translation initiation site of said mutant allele.

41. A method for reducing the level of an RNA or the level of a polypeptide in a mammal having a mutant allele that causes a dominant disorder, wherein said RNA and said polypeptide are encoded by said mutant allele, and wherein said mammal is heterozygous for said mutant allele, said method comprising administering at least two polyamide nucleic acid oligomers to said mammal under conditions wherein the level of said RNA is reduced to a greater extent than the reduction, if any, in the amount of a second RNA or the level of said polypeptide is reduced to a greater extent than the reduction, if any, in the amount of a second polypeptide, wherein each of said at least two polyamide nucleic acid oligomers has a different sequence, wherein said second RNA and said second polypeptide are encoded by a second allele in said mammal, and wherein said second allele corresponds to said mutant allele and does not cause said dominant disorder.

42. The method of claim 41, wherein said mammal is a human.

43. The method of claim 41, wherein said dominant disorder is an autosomal dominant disorder.

44. The method of claim 41, wherein said dominant disorder is Huntington disease.

45. The method of claim 41, wherein said RNA is mRNA.

46. The method of claim 41, wherein each of said at least two polyamide nucleic acid oligomers is administered into the brain of said mammal.

47. The method of claim 41, wherein each of said at least two polyamide nucleic acid oligomers is administered intraperitoneally to said mammal.

48. The method of claim 41, wherein each of said at least two polyamide nucleic acid oligomers comprises a sequence having specificity for a transcription initiation site of said mutant allele, a translation initiation site of said mutant allele, or a region between said transcription initiation site and said translation initiation site of said mutant allele.

49. A method for reducing the level of an RNA or the level of a polypeptide in a mammal having a mutant allele that causes a dominant disorder, wherein said RNA and said polypeptide are encoded by said mutant allele, and wherein said mammal is heterozygous for said mutant allele, said method comprising administering, to said mammal, between 0.05 mg and 0.5 mg of a polyamide nucleic acid oligomer per kg of body weight of said mammal, said administration being under conditions wherein the level of said RNA is reduced to a greater extent than the reduction, if any, in the amount of a second RNA or the level of said polypeptide is reduced to a greater extent than the reduction, if any, in the amount of a second polypeptide, wherein said second RNA and said second polypeptide are encoded by a second allele in said mammal, and wherein said second allele corresponds to said mutant allele and does not cause said dominant disorder.

50. The method of claim 49, wherein said mammal is a human.

51. The method of claim 49, wherein said dominant disorder is an autosomal dominant disorder.

52. The method of claim 49, wherein said dominant disorder is Huntington disease.

53. The method of claim 49, wherein said RNA is mRNA.

54. The method of claim 49, wherein said polyamide nucleic acid oligomer is administered into the brain of said mammal.

55. The method of claim 49, wherein said polyamide nucleic acid oligomer is administered intraperitoneally to said mammal.

56. The method of claim 49, wherein said polyamide nucleic acid oligomer comprises a sequence having specificity for a transcription initiation site of said mutant allele, a translation initiation site of said mutant allele, or a region between said transcription initiation site and said translation initiation site of said mutant allele.

57. The method of claim 49, wherein said polyamide nucleic acid oligomer comprises the sequence set forth in SEQ ID NO:3.

58. A kit for assisting a medical professional in treating multiple different mammals, wherein each of said multiple different mammals has a dominant disorder caused by a mutant allele, said kit comprising a plurality of polyamide nucleic acid oligomers, wherein the sequence of each of said plurality of polyamide nucleic acid oligomers is different and based on sequence information obtained from each of said multiple different mammals, wherein at least one of said plurality of polyamide nucleic acid oligomers has specificity for said mutant allele from each of said multiple different mammals such that administration of said at least one of said plurality of polyamide nucleic acid oligomers to each of said multiple different mammals reduces expression of a first polypeptide in each of said multiple different mammals, wherein the amount of reduction in expression of said first polypeptide is greater than the amount of reduction, if any, in expression of a second polypeptide, wherein said first polypeptide is encoded by said mutant allele, wherein said second polypeptide is encoded by a second allele in each of said multiple different mammals, and wherein said second allele corresponds to said mutant allele and does not cause said dominant disorder.

59. The kit of claim 58, wherein each of said multiple different mammals is a human.

60. The kit of claim 58, wherein said dominant disorder is an autosomal dominant disorder.

61. The kit of claim 58, wherein said dominant disorder is Huntington disease.

62. The kit of claim 58, wherein said sequence information was obtained by PCR.

63. The kit of claim 58, wherein each of said plurality of polyamide nucleic acid oligomers comprises a sequence having specificity for a transcription initiation site of said mutant allele, a translation initiation site of said mutant allele, or a region between said transcription initiation site and said translation initiation site of said mutant allele.


SEQUENCE LISTING (4 sequences)

SEQ ID NO: 1 - DNA, 174 bp, Homo Sapien
cccattcattgccccggtgctgagcggcgccgcgagtcggcccgaggcctccggggactgccgtgccgggcgggagaccgccatggcgaccctggaaaagctgatgaaggccttcgagtccctcaagtccttccagcagcagcagcagcagcagcagcagcagcagcagcagca

SEQ ID NO: 2 - DNA, 181 bp, Mus musculus
cccattcattgccttgctgctaagtggcgccgcgtagtgccagtaggctccaagtcttcagggtctgtcccatcgggcaggaagccgtcatggcaaccctggaaaagctgatgaaggctttcgactcgctcaagtcgtttcagcagcaacagcagcagcagccaccgccgcaggcgccgcc

SEQ ID NO: 3 - DNA, 14 bp, Artificial Sequence
  Synthetically generated oligonucleotide
ggactgccgtgccg

SEQ ID NO: 4 - DNA, 14 bp, Artificial Sequence
  Synthetically generated oligonucleotide
gcagcggcggtcct